SEQUENCE LISTING (50 sequences)

SEQ ID NO: 1 - RNA, 21 nt, Artificial Sequence
  Description of Artificial Sequence Synthetic oligonucleotide
  modified_base (20)..(21): 2′-O-Methyl nucleotide
ugaugaagauuaaaaccuuuu

SEQ ID NO: 2 - RNA, 21 nt, Artificial Sequence
  Description of Artificial Sequence Synthetic oligonucleotide
  modified_base (20)..(21): 2′-O-Methyl nucleotide
ugaugaagauuaagaaaaauu

SEQ ID NO: 3 - RNA, 21 nt, Artificial Sequence
  Description of Artificial Sequence Synthetic oligonucleotide
  modified_base (20)..(21): 2′-O-Methyl nucleotide
augaagauuaaaaccuucauu

SEQ ID NO: 4 - RNA, 21 nt, Artificial Sequence
  Description of Artificial Sequence Synthetic oligonucleotide
  modified_base (20)..(21): 2′-O-Methyl nucleotide
gaugaagauuaaaaccuucuu

SEQ ID NO: 5 - RNA, 21 nt, Artificial Sequence
  Description of Artificial Sequence Synthetic oligonucleotide
  modified_base (20)..(21): 2′-O-Methyl nucleotide
gaagauuaaaaccuucaucuu

SEQ ID NO: 6 - RNA, 21 nt, Artificial Sequence
  Description of Artificial Sequence Synthetic oligonucleotide
  modified_base (20)..(21): 2′-O-Methyl nucleotide
ugaagauuaaaaccuucauuu

SEQ ID NO: 7 - RNA, 21 nt, Artificial Sequence
  Description of Artificial Sequence Synthetic oligonucleotide
  modified_base (20)..(21): 2′-O-Methyl nucleotide
ugaggaagauuaagaaaaauu

SEQ ID NO: 8 - RNA, 21 nt, Artificial Sequence
  Description of Artificial Sequence Synthetic oligonucleotide
  modified_base (20)..(21): 2′-O-Methyl nucleotide
uugaugaagauuaagaaaauu

SEQ ID NO: 9 - RNA, 21 nt, Artificial Sequence
  Description of Artificial Sequence Synthetic oligonucleotide
  modified_base (20)..(21): 2′-O-Methyl nucleotide
gaugaagauuaagaaaaaguu

SEQ ID NO: 10 - RNA, 21 nt, Artificial Sequence
  Description of Artificial Sequence Synthetic oligonucleotide
  modified_base (20)..(21): 2′-O-Methyl nucleotide
augaugaagauuaaaaccuuu

SEQ ID NO: 11 - RNA, 21 nt, Artificial Sequence
  Description of Artificial Sequence Synthetic oligonucleotide
  modified_base (20)..(21): 2′-O-Methyl nucleotide
aagauuaaaaccuucaucauu

SEQ ID NO: 12 - RNA, 21 nt, Artificial Sequence
  Description of Artificial Sequence Synthetic oligonucleotide
  modified_base (20)..(21): 2′-O-Methyl nucleotide
uuuuccagaauguuauaaauu

SEQ ID NO: 13 - RNA, 21 nt, Artificial Sequence
  Description of Artificial Sequence Synthetic oligonucleotide
  modified_base (20)..(21): 2′-O-Methyl nucleotide
auuuuccagaauguuauaauu

SEQ ID NO: 14 - RNA, 21 nt, Artificial Sequence
  Description of Artificial Sequence Synthetic oligonucleotide
  modified_base (20)..(21): 2′-O-Methyl nucleotide
uuccagaauguuauaaauuuu

SEQ ID NO: 15 - RNA, 21 nt, Artificial Sequence
  Description of Artificial Sequence Synthetic oligonucleotide
  modified_base (20)..(21): 2′-O-Methyl nucleotide
uuuccagaauguuauaaauuu

SEQ ID NO: 16 - RNA, 21 nt, Artificial Sequence
  Description of Artificial Sequence Synthetic oligonucleotide
  modified_base (20)..(21): 2′-O-Methyl nucleotide
agauuaaaaccuucaucauuu

SEQ ID NO: 17 - RNA, 21 nt, Artificial Sequence
  Description of Artificial Sequence Synthetic oligonucleotide
  modified_base (20)..(21): 2′-O-Methyl nucleotide
ccuuuauggauaguauaucuu

SEQ ID NO: 18 - RNA, 21 nt, Artificial Sequence
  Description of Artificial Sequence Synthetic oligonucleotide
  modified_base (20)..(21): 2′-O-Methyl nucleotide
uuuguaacugauuuagagauu

SEQ ID NO: 19 - RNA, 21 nt, Artificial Sequence
  Description of Artificial Sequence Synthetic oligonucleotide
  modified_base (20)..(21): 2′-O-Methyl nucleotide
uuguaacugauuuagagaauu

SEQ ID NO: 20 - RNA, 21 nt, Artificial Sequence
  Description of Artificial Sequence Synthetic oligonucleotide
  modified_base (20)..(21): 2′-O-Methyl nucleotide
aacuaaugaugaagauuaauu

SEQ ID NO: 21 - RNA, 21 nt, Artificial Sequence
  Description of Artificial Sequence Synthetic oligonucleotide
  modified_base (20)..(21): 2′-O-Methyl nucleotide
aaacuaaugaugaagauuauu

SEQ ID NO: 22 - RNA, 21 nt, Artificial Sequence
  Description of Artificial Sequence Synthetic oligonucleotide
  modified_base (20)..(21): 2′-O-Methyl nucleotide
uccucgacacgaaugcaaauu

SEQ ID NO: 23 - RNA, 21 nt, Artificial Sequence
  Description of Artificial Sequence Synthetic oligonucleotide
  modified_base (20)..(21): 2′-O-Methyl nucleotide
gcaauucauuggacauuauuu

SEQ ID NO: 24 - RNA, 21 nt, Artificial Sequence
  Description of Artificial Sequence Synthetic oligonucleotide
  modified_base (20)..(21): 2′-O-Methyl nucleotide
guacgaagcuguacauaaauu

SEQ ID NO: 25 - RNA, 21 nt, Artificial Sequence
  Description of Artificial Sequence Synthetic oligonucleotide
  modified_base (20)..(21): 2′-O-Methyl nucleotide
gauuauguccgguuauguauu

SEQ ID NO: 26 - RNA, 21 nt, Artificial Sequence
  Description of Artificial Sequence Synthetic oligonucleotide
  modified_base (20)..(21): 2′-O-Methyl nucleotide
aagguuuuaaucuucaucauu

SEQ ID NO: 27 - RNA, 21 nt, Artificial Sequence
  Description of Artificial Sequence Synthetic oligonucleotide
  modified_base (20)..(21): 2′-O-Methyl nucleotide
uuuuucuuaaucuucaucauu

SEQ ID NO: 28 - RNA, 21 nt, Artificial Sequence
  Description of Artificial Sequence Synthetic oligonucleotide
  modified_base (20)..(21): 2′-O-Methyl nucleotide
ugaagguuuuaaucuucauuu

SEQ ID NO: 29 - RNA, 21 nt, Artificial Sequence
  Description of Artificial Sequence Synthetic oligonucleotide
  modified_base (20)..(21): 2′-O-Methyl nucleotide
gaagguuuuaaucuucaucuu

SEQ ID NO: 30 - RNA, 21 nt, Artificial Sequence
  Description of Artificial Sequence Synthetic oligonucleotide
  modified_base (20)..(21): 2′-O-Methyl nucleotide
gaugaagguuuuaaucuucuu

SEQ ID NO: 31 - RNA, 21 nt, Artificial Sequence
  Description of Artificial Sequence Synthetic oligonucleotide
  modified_base (20)..(21): 2′-O-Methyl nucleotide
augaagguuuuaaucuucauu

SEQ ID NO: 32 - RNA, 21 nt, Artificial Sequence
  Description of Artificial Sequence Synthetic oligonucleotide
  modified_base (20)..(21): 2′-O-Methyl nucleotide
uuuuucuuaaucuuccucauu

SEQ ID NO: 33 - RNA, 21 nt, Artificial Sequence
  Description of Artificial Sequence Synthetic oligonucleotide
  modified_base (20)..(21): 2′-O-Methyl nucleotide
uuuucuuaaucuucaucaauu

SEQ ID NO: 34 - RNA, 21 nt, Artificial Sequence
  Description of Artificial Sequence Synthetic oligonucleotide
  modified_base (20)..(21): 2′-O-Methyl nucleotide
cuuuuucuuaaucuucaucuu

SEQ ID NO: 35 - RNA, 21 nt, Artificial Sequence
  Description of Artificial Sequence Synthetic oligonucleotide
  modified_base (20)..(21): 2′-O-Methyl nucleotide
agguuuuaaucuucaucauuu

SEQ ID NO: 36 - RNA, 21 nt, Artificial Sequence
  Description of Artificial Sequence Synthetic oligonucleotide
  modified_base (20)..(21): 2′-O-Methyl nucleotide
ugaugaagguuuuaaucuuuu

SEQ ID NO: 37 - RNA, 21 nt, Artificial Sequence
  Description of Artificial Sequence Synthetic oligonucleotide
  modified_base (20)..(21): 2′-O-Methyl nucleotide
uuuauaacauucuggaaaauu

SEQ ID NO: 38 - RNA, 21 nt, Artificial Sequence
  Description of Artificial Sequence Synthetic oligonucleotide
  modified_base (20)..(21): 2′-O-Methyl nucleotide
uuauaacauucuggaaaauuu

SEQ ID NO: 39 - RNA, 21 nt, Artificial Sequence
  Description of Artificial Sequence Synthetic oligonucleotide
  modified_base (20)..(21): 2′-O-Methyl nucleotide
aauuuauaacauucuggaauu

SEQ ID NO: 40 - RNA, 21 nt, Artificial Sequence
  Description of Artificial Sequence Synthetic oligonucleotide
  modified_base (20)..(21): 2′-O-Methyl nucleotide
auuuauaacauucuggaaauu

SEQ ID NO: 41 - RNA, 21 nt, Artificial Sequence
  Description of Artificial Sequence Synthetic oligonucleotide
  modified_base (20)..(21): 2′-O-Methyl nucleotide
augaugaagguuuuaaucuuu

SEQ ID NO: 42 - RNA, 21 nt, Artificial Sequence
  Description of Artificial Sequence Synthetic oligonucleotide
  modified_base (20)..(21): 2′-O-Methyl nucleotide
gauauacuauccauaaagguu

SEQ ID NO: 43 - RNA, 21 nt, Artificial Sequence
  Description of Artificial Sequence Synthetic oligonucleotide
  modified_base (20)..(21): 2′-O-Methyl nucleotide
ucucuaaaucaguuacaaauu

SEQ ID NO: 44 - RNA, 21 nt, Artificial Sequence
  Description of Artificial Sequence Synthetic oligonucleotide
  modified_base (20)..(21): 2′-O-Methyl nucleotide
uucucuaaaucaguuacaauu

SEQ ID NO: 45 - RNA, 21 nt, Artificial Sequence
  Description of Artificial Sequence Synthetic oligonucleotide
  modified_base (20)..(21): 2′-O-Methyl nucleotide
uuaaucuucaucauuaguuuu

SEQ ID NO: 46 - RNA, 21 nt, Artificial Sequence
  Description of Artificial Sequence Synthetic oligonucleotide
  modified_base (20)..(21): 2′-O-Methyl nucleotide
uaaucuucaucauuaguuuuu

SEQ ID NO: 47 - RNA, 21 nt, Artificial Sequence
  Description of Artificial Sequence Synthetic oligonucleotide
  modified_base (20)..(21): 2′-O-Methyl nucleotide
uuugcauucgugucgaggauu

SEQ ID NO: 48 - RNA, 21 nt, Artificial Sequence
  Description of Artificial Sequence Synthetic oligonucleotide
  modified_base (20)..(21): 2′-O-Methyl nucleotide
auaauguccaaugaauugcuu

SEQ ID NO: 49 - RNA, 21 nt, Artificial Sequence
  Description of Artificial Sequence Synthetic oligonucleotide
  modified_base (20)..(21): 2′-O-Methyl nucleotide
uuuauguacagcuucguacuu

SEQ ID NO: 50 - RNA, 21 nt, Artificial Sequence
  Description of Artificial Sequence Synthetic oligonucleotide
  modified_base (20)..(21): 2′-O-Methyl nucleotide
uacauaaccggacauaaucuu

(19) United States
(12) Patent Application Publication
(10) Pub. No.: US 2018/0235995 A1
(43) Pub. Date: Aug. 23, 2018

(54) RNA INTERFERENCE THERAPEUTICS AGAINST EBOLA VIRUS

(71) Applicants: Wenbin Ying, (US); Nitto Denko Corporation, Osaka (JP)

(72) Inventors: Wenbin Ying, Oceanside, CA (US); Bharat Majeti, San Diego, CA (US); Jens Harborth, Carlsbad, CA (US); Roger Adami, Carlsbad, CA (US)

(21) Appl. No.: 15/551,270

(22) PCT Filed: Feb. 18, 2016
(86) PCT No.: PCT/US16/18472

§ 371 Date: Aug. 15, 2017

Related U.S. Application Data
(60) Provisional application No. 62/118,436, filed on Feb. 19, 2015.

(51) Int. Cl. A61K 31/713; A61K 9/00; A61K 9/127; A61P 31/14

(52) U.S. Cl. CPC A61K 31/713; A61P 31/14; A61K 9/1271; A61K 9/0019

(57) ABSTRACT

This invention provides methods and compositions for inhibiting Ebola virus and Ebola infection utilizing gene silencing siRNAs. The siRNAs can be targeted to one or more Ebola genes including NP, VP30, VP35, L polymerase, glycoprotein (GP), VP24, VP40, and intergenic overlapping regions selected from NP/VP35, VP35/VP40, GP/VP30, and VP24/L. A composition containing the siRNAs can be administered in a delivery vehicle such as liposomes.

### GOVERNMENT LICENSE RIGHTS

This invention was made with government support under a cooperative agreement awarded by the United States Army Medical Research Institute of Infectious Diseases. The government of the United States of America has certain rights in the invention.

### SEQUENCE LISTING

This application includes a Sequence Listing submitted electronically as an ASCII file created on Feb. 2, 2016, named ND6020642WO_SL.txt, which is 16,017 bytes in size, and is hereby incorporated by reference in its entirety.

### TECHNICAL FIELD OF THE INVENTION

This invention relates to the fields of biopharmaceuticals and therapeutics composed of nucleic acid based molecules. More particularly, this invention relates to methods and compositions utilizing RNA interference for preventing, treating or ameliorating infections caused by Ebola virus.

### BACKGROUND OF THE INVENTION

Ebola virus disease, also known as Ebola haemorrhagic fever, is a severe, often fatal illness in humans. The virus is transmitted to people from wild animals and spreads in the human population through human-to-human transmission. The average Ebola virus disease fatality rate currently is about 50%, and has been as high as 90%. Ebola spreads through human-to-human transmission via direct contact, through broken skin or mucous membranes, with the blood, secretions, organs or other bodily fluids of infected people, and with surfaces and materials, e.g. bedding, clothing, contaminated with these fluids.

The virus family Filoviridae includes three genera: Cuevavirus, Marburgvirus, and Ebolavirus. There are five species that have been identified: Zaire, Bundibugyo, Sudan, Reston and Taï Forest. The first three, Zaire ebolavirus (ZEBOV), Bundibugyo ebolavirus, and Sudan ebolavirus have been associated with large outbreaks in Africa. The virus causing the 2014 west Africa outbreak belongs to the Zaire species.

Ebola virus is a nonsegmented negative-strand (NNS) RNA virus. The RNA negative-strand genome provides nucleocapsid proteins NP, VP30, VP35, and an RNA-dependent RNA polymerase L to form a ribonucleoprotein complex for viral replication and transcription. Also provided are membrane-associated proteins glycoprotein (GP), VP24, and VP40. Replication and transcription of Ebola virus occurs in the cytoplasm. After entry into the cytosol, the ribonucleoprotein complex-associated genome is uncoated and exposed to the RNA-dependent RNA polymerase to be transcribed into a short, uncapped, positive-stranded RNA leader and seven-capped and polyadenylated mRNAs, which encode the viral proteins necessary for replication.

What is needed are compositions and methods for preventing or treating Ebola virus infection. There is a continuing need for RNA molecules, structures and compositions for preventing or treating Ebola virus infection.

### BRIEF SUMMARY

This invention relates to methods and compositions for nucleic acid based therapeutic compounds against Ebola virus. In some embodiments, this invention provides interfering RNA molecules, structures and compositions that can silence Ebola viral expression. The structures and compositions of this disclosure can be used in preventing or treating Ebola virus infections.

This invention can provide compositions for delivery of therapeutic RNA molecules, as well as methods of use thereof. The RNA-based compositions of this invention can be used in methods for preventing or treating Ebola virus infection.

Embodiments of this invention include the following:

A composition for gene silencing Ebola virus comprising one or more siRNAs targeted to at least one Ebola virus gene selected from NP, VP30, VP35, L polymerase, glycoprotein (GP), VP24, VP40, and intergenic overlapping regions selected from NP/VP35, VP35/VP40, GP/VP30, and VP24/L.

The composition above, wherein the one or more siRNAs are targeted to at least two Ebola genes selected from NP, VP30, VP35, L polymerase, glycoprotein (GP), VP24, VP40, and intergenic overlapping regions selected from NP/VP35, VP35/VP40, GPNP30, and VP24/L.

The composition above, wherein the one or more siRNAs have at least 60% sequence homology as compared to all 158 known and sequenced strains of Ebola virus.

The composition above, wherein the one or more siRNAs have 100% sequence homology as compared to the Ebola Guinea strains found in year 2014 in western Africa.

The composition above, wherein at least one of the siRNAs inhibits an Ebola virus gene with an IC50 of less than 20 nM, or less than 10 nM. In some embodiments, a single administration of at least one of the siRNAs inhibits Ebola virus titer levels by at least 25% in vivo.

The siRNAs may comprise one or more modified, non-natural, or chemically-modified nucleotides in the double-stranded region, such as one or more 2′-OMe nucleotides. The siRNAs may comprise a blunt end, or a 3′ overhang.

The composition may further include a delivery vehicle, and the siRNAs can be encapsulated in a liposome. In some embodiments, a delivery vehicle can include an ionizable lipid, a structural lipid, one or more stabilizer lipids, and a lipid for reducing immunogenicity.

This invention further contemplates methods for silencing Ebola virus gene expression in a cell, the method comprising contacting the cell with a siRNA above. Additional methods include:

A method for silencing Ebola virus gene expression in a mammal in need thereof, the method comprising administering to the mammal a composition above.

A method for preventing, treating or ameliorating one or more symptoms of Ebola virus infection in a mammal in need thereof, the method comprising administering to the mammal a therapeutically effective amount of a composition above.

A method for preventing, treating or ameliorating one or more symptoms of Ebola virus infection in a mammal in need thereof, the method comprising administering to the mammal a therapeutically effective amount of a composition above, wherein after administration the Ebola viral titer of the mammal is reduced by at least 90%.

A method for inhibiting the replication of Ebola virus in a mammal in need thereof, the method comprising administering to the mammal a therapeutically effective amount of a composition above.

### DETAILED DESCRIPTION OF THE INVENTION

This invention relates to methods and compositions for nucleic acid based therapeutic compounds against Ebola virus. The Ebola virus can be attacked by compositions of this invention that can regulate or silence viral expression products.

In some embodiments, this invention provides interfering RNA molecules, structures and compositions that can silence Ebola viral expression.

The structures and compositions of this disclosure can be used in preventing or treating Ebola virus infections.

In further embodiments, this invention provides compositions for delivery and uptake of one or more therapeutic RNA molecules of this invention, as well as methods of use thereof. The RNA-based compositions of this invention can be used in methods for preventing or treating Ebola virus infection.

The Ebola virus Zaire species, Mayinga isolate, is GenBank accession no. AY354458. Zaire Ebola virus isolate Ebola virus/H.sapiens-tc/COD/1976/Yambuku-Mayinga, complete genome is found at NCBI Reference Sequence: NC_002549.1.

The RNA-based compositions of this invention can be used in methods for preventing or treating Ebola virus infection based on Ebola Zaire Kikwit strain.

Therapeutic compositions of this invention include nucleic acid molecules that are active in RNA interference. The therapeutic nucleic acid molecules of this invention are targeted to Ebola virus for gene silencing.

In some embodiments, this invention provides small interfering RNAs (siRNAs) that can regulate or silence ZEBOV gene expression.

The siRNAs of this invention can be used for preventing and/or treating Ebola virus infections.

Embodiments of this invention can further provide a vehicle, formulation, or lipid nanoparticle formulation for delivery of the inventive siRNAs to subjects in need of preventing and/or treating Ebola virus infection. This invention further contemplates methods for administering siRNAs and other therapeutics to mammals.

A nanoparticle of this disclosure can range in size from 20 nm to 200 nm.

In some embodiments, a composition for delivery of a siRNA can include a delivery vehicle for in vivo delivery of the siRNA.

In some embodiments, this invention provides double-stranded ribonucleic acid agents (dsRNAs), which are short interfering RNAs (siRNAs), that can be used in mediating RNA interference to inhibit the expression of one or two genes of the Ebola virus.

In certain aspects, a dsRNA or composition of this invention can be used to treat pathological processes mediated by Ebola infection.

The therapeutic molecules and formulations of this invention can be used for RNA interference of one or more of Ebola virus genes providing NP, VP30, VP35, L polymerase, glycoprotein (GP), VP24, and VP40.

In certain embodiments, a therapeutic composition of this invention can contain two anti-Ebola siRNAs that are targeted to highly conserved regions of VP35, L polymerase, or intergenic overlapping regions, such as NP/VP35, VP35/VP40, GP/VP30, and VP24/L, and which have sequence homology with 82% to 100% of all sequenced strains of Ebola, and 100% homology to the 2014 Guinea strains epidemic in West Africa.

The therapeutic molecules and formulations of this invention can be used for silencing or inhibiting the replication of Ebola virus in vitro and in vivo.

In certain embodiments, a combination of therapeutic molecules of this invention can be used for silencing or inhibiting the replication of Ebola virus. A combination of therapeutic molecules of this invention can be targeted for RNA interference of any combination of Ebola virus genes providing NP, VP30, VP35, L polymerase, glycoprotein (GP), VP24, and VP40.

In some embodiments, each strand of a siRNA molecule of this invention can be from 15 to 60 nucleotides in length, or from 15 to 40 nucleotides in length, or from 19 to 25 nucleotides in length.

In some aspects, a therapeutic molecule of this invention can include modified, non-natural, or chemically-modified analog nucleotides.

In certain embodiments, a therapeutic molecule of this invention can be modified with deoxynucleotides, 2′-OMe nucleotides, or 2′F-modified nucleotides.

In further aspects, a therapeutic molecule of this invention can include a 3′ overhang of 1 to 6 nucleotides, which may contain deoxythymidine (dT) nucleotides or modified, non-natural, or chemically-modified analog nucleotides.

A nucleic acid molecule for inhibiting Ebola virus can contain a sense strand and an antisense strand, wherein the strands form a duplex region. The nucleic acid molecules can be siRNA molecules for inhibiting expression of one or more Ebola virus genes, and may contain one or more nucleotides that are modified or chemically-modified.

In some embodiments, the nucleic acid siRNA molecules for inhibiting Ebola virus may include 2′-deoxy nucleotides, 2′-O-alkyl substituted nucleotides, 2′-deoxy-2′-fluoro substituted nucleotides, or any combination thereof. In certain embodiments, the 2′-deoxy nucleotides may be in the seed region of the siRNA molecules. In certain aspects, the siRNA molecules for inhibiting Ebola virus may have deoxynucleotides in a plurality of positions in the antisense strand.

The nucleic acid molecules of this invention may advantageously inhibit expression of Ebola virus with an IC50 of less than 50 nM, or less than 20 nM, or less than 10 nM. In certain embodiments, the nucleic acid molecules may inhibit expression of Ebola virus titer levels by at least 25% in vivo, upon a single administration of the molecules.

In additional aspects, a therapeutic duplex molecule of this invention can have a blunt end.

In some embodiments, an inventive siRNA can have a sense strand and an antisense strand, which are in general complementary. In certain embodiments, the antisense strand can have a sequence that is partially, substantially or 100% identical to a target sequence of an Ebola virus. An antisense strand of an inventive siRNA can have, or consist of, from 12 to 19 contiguous nucleotides of a target sequence of an Ebola virus.

An siRNA of this invention is capable of mediating target-specific gene silencing of an Ebola virus gene, including any one of L polymerase, VP24, VP30, VP35, VP40, NP, and GP.

A therapeutic formulation of this invention for the delivery of one or more molecules active for Ebola virus gene silencing can be administered to a mammal in need thereof. A therapeutically effective amount of the formulation and active agent, which may be encapsulated in a liposome, can be administered to a mammal for preventing or treating Ebola virus infection.

A therapeutically-effective formulation of this invention can be administered by various routes, including intravenous, intraperitoneal, intramuscular, subcutaneous, and oral.

A therapeutically-effective formulation of this invention can be administered by systemic delivery that can provide a broad biodistribution of the active agent.

Embodiments of this invention can provide a therapeutic formulation, which includes an inventive therapeutic molecule and a pharmaceutically-acceptable carrier.

In some embodiments, the replication of Ebola virus can be inhibited, regulated, inactivated or silenced by methods of this invention for preventing or treating Ebola virus infection.

Methods of this invention can involve knockdown of an Ebola virus gene expression, either in vitro or in vivo.

The methods and compositions of this invention can be used to prevent or treat Ebola virus infection in mammals, nonhuman primates, and human subjects.

Embodiments of this invention may further provide methods for preventing, treating or ameliorating one or more symptoms of Ebola infection, or reducing the risk of developing Ebola infection, or delaying the onset of Ebola infection in a mammal in need thereof.

Embodiments of this invention further provide pharmaceutical compositions containing the siRNA molecules and a pharmaceutically acceptable carrier. In some embodiments, the carrier may be a lipid molecule, or liposome. This invention includes vectors or cells comprising the nucleic acid molecules.

In certain aspects, the methods of this invention can be used for inhibiting the replication of Ebola virus in a mammal in need thereof by administering to the mammal a therapeutically-effective amount of a dsRNA formulation or formulations containing active RNAs targeted to one or more Ebola virus genes.

Administering a dsRNA formulation of this invention may reduce Ebola viral titer in a subject by 20% to 100%.

A formulation of this invention may be used in combination with an anti-viral drug.

An effective dose of a formulation of this invention may be administered from 1 to 20 times per day, or once per week after exposure to Ebola virus. The duration of administration can be 1, 2, 3, 4, 5, 6 or 7 days, or can be 1, 2, 3, 4, 5, 6, 8, 10 or 12 weeks.

Designations that may be used herein include mA, mG, mC, and mU, which refer to the 2′-O-Methyl modified ribonucleotides.

### Modified and Chemically-Modified siRNAs

Embodiments of this invention encompass siRNA molecules that are modified or chemically-modified to provide enhanced properties for therapeutic use, such as increased activity and potency for gene silencing. This invention provides modified or chemically-modified siRNA molecules that can have increased serum stability, as well as reduced off target effects, without loss of activity and potency of the siRNA molecules for gene modulation and gene silencing. In some aspects, this invention provides siRNAs having modifications or chemical modifications in various combinations, which enhance the stability and efficacy of the siRNA.

In some embodiments, the siRNA molecules of this invention can have passenger strand off target activity reduced by at least 10-fold, or at least 20-fold, or at least 30-fold, or at least 50-fold, or at least 100-fold.

As used herein, the terms modified and chemically-modified refer to changes made in the structure of a naturally-occurring nucleotide or nuclei acid structure of an siRNA, which encompasses siRNAs having one or more nucleotide analogs, altered nucleotides, non-standard nucleotides, non-naturally occurring nucleotides, and combinations thereof.

In some embodiments, the number of modified or chemically-modified structures in an siRNA can include all of the structural components, and/or all of the nucleotides of the siRNA molecule.

Examples of modified and chemically-modified siRNAs include siRNAs having modification of the sugar group of a nucleotide, modification of a nucleobase of a nucleotide, modification of a nucleic acid backbone or linkage, modification of the structure of a nucleotide or nucleotides at the terminus of a siRNA strand, and combinations thereof.

Examples of modified and chemically-modified siRNAs include siRNAs having modification of the substituent at the 2′ carbon of the sugar.

Examples of modified and chemically-modified siRNAs include siRNAs having modification at the 5′ end, the 3′ end, or at both ends of a strand.

Examples of modified and chemically-modified siRNAs include siRNAs having modifications that produce complementarity mismatches between the strands.

Examples of modified and chemically-modified siRNAs include siRNAs having a 5′-propylamine end, a 5′-phosphorylated end, a 3′-puromycin end, or a 3′-biotin end group.

Examples of modified and chemically-modified siRNAs include siRNAs having a 2′-fluoro substituted ribonucleotide, a 2′-OMe substituted ribonucleotide, a 2′-deoxy ribonucleotide, a 2′-amino substituted ribonucleotide, a 2′-thio substituted ribonucleotide.

Examples of modified and chemically-modified siRNAs include siRNAs having one or more 5-halouridines, 5-halocytidines, 5-methylcytidines, ribothymidines, 2-aminopurines, 2,6-diaminopurines, 4-thiouridines, or 5-aminoallyluridines.

Examples of modified and chemically-modified siRNAs include siRNAs having one or more phosphorothioate groups.

Examples of modified and chemically-modified siRNAs include siRNAs having one or more 2′-fluoro substituted ribonucleotides, 2′-fluorouridines, 2′-fluorocytidines, 2′-deoxyribonucleotides, 2′-deoxyadenosines, or 2′-deoxyguanosines.

Examples of modified and chemically-modified siRNAs include siRNAs having one or more phosphorothioate linkages.

Examples of modified and chemically-modified siRNAs include siRNAs having one or more alkylene diol linkages, oxy-alkylthio linkages, or oxycarbonyloxy linkages.

Examples of modified and chemically-modified siRNAs include siRNAs having one or more deoxyabasic groups, inosines, N3-methyl-uridines, N6,N6-dimethyl-adenosines, pseudouridines, purine ribonucleosides, and ribavirins.

Examples of modified and chemically-modified siRNAs include siRNAs having one or more 3′ or 5′ inverted terminal groups.

Examples of modified and chemically-modified siRNAs include siRNAs having one or more 5-(2-amino)propyluridines, 5-bromouridines, adenosines, 8-bromo guanosines, 7-deaza-adenosines, or N6-methyl adenosine.

### RNA Interference

RNA interference (RNAi) refers to sequence-specific post-transcriptional gene silencing in animals mediated by short interfering RNAs (siRNAs). See, e.g., Zamore et al., Cell, 2000, Vol. 101, pp. 25-33; Fire et al., Nature, 1998, Vol. 391, pp. 806811; Sharp, Genes & Development, 1999, Vol. 13, pp. 139-141.

An RNAi response in cells can be triggered by a double stranded RNA (dsRNA), although the mechanism is not yet fully understood. Certain dsRNAs in cells can undergo the action of Dicer enzyme, a ribonuclease III enzyme. See, e.g., Zamore et al., Cell, 2000, Vol. 101, pp. 25-33; Hammond et al., Nature, 2000, Vol. 404, pp. 293-296. Dicer can process the dsRNA into shorter pieces of dsRNA, which are siRNAs.

In general, siRNAs can be from about 21 to about 23 nucleotides in length and include a base pair duplex region about 19 nucleotides in length.

RNAi involves an endonuclease complex known as the RNA induced silencing complex (RISC). An siRNA has an antisense or guide strand which enters the RISC complex and mediates cleavage of a single stranded RNA target having a sequence complementary to the antisense strand of the siRNA duplex. The other strand of the siRNA is the passenger strand. Cleavage of the target RNA takes place in the middle of the region complementary to the antisense strand of the siRNA duplex See, e.g., Elbashir et al., Genes & Development, 2001, Vol. 15, pp. 188-200.

As used herein, the term “sense strand” refers to a nucleotide sequence of a siRNA molecule that is partially or fully complementary to at least a portion of a corresponding antisense strand of the siRNA molecule. The sense strand of a siRNA molecule can include a nucleic acid sequence having homology with a target nucleic acid sequence.

As used herein, the term “antisense strand” refers to a nucleotide sequence of a siRNA molecule that is partially or fully complementary to at least a portion of a target nucleic acid sequence. The antisense strand of a siRNA molecule can include a nucleic acid sequence that is complementary to at least a portion of a corresponding sense strand of the siRNA molecule.

RNAi molecules can down regulate or knock down gene expression by mediating RNA interference in a sequence-specific manner. See, e.g., Zamore et al., Cell, 2000, Vol. 101, pp. 25-33; Elbashir et al., Nature, 2001, Vol. 411, pp. 494-498; Kreutzer et al., WO2000/044895; Zernicka-Goetz et al., WO2001/36646; Fire et al., WO1999/032619; Plaetinck et al., WO2000/01846; Mello et al., WO2001/029058.

As used herein, the terms “inhibit,” “down-regulate,” or “reduce” with respect to gene expression means that the expression of the gene, or the level of mRNA molecules encoding one or more proteins, or the activity of one or more of the encoded proteins is reduced below that observed in the absence of a RNAi molecule or siRNA of this invention. For example, the level of expression, level of mRNA, or level of encoded protein activity may be reduced by at least 1%, or at least 10%, or at least 20%, or at least 50%, or at least 90%, or more from that observed in the absence of a RNAi molecule or siRNA of this invention.

RNAi molecules can also be used to knock down viral gene expression, and therefore affect viral replication.

RNAi molecules can be made from separate polynucleotide strands: a sense strand or passenger strand, and an antisense strand or guide strand. The guide and passenger strands are at least partially complementary. The guide strand and passenger strand can form a duplex region having from about 15 to about 49 base pairs.

In some embodiments, the duplex region of a siRNA can have 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 base pairs.

In certain embodiments, a RNAi molecule can be active in a RISC complex, with a length of duplex region active for RISC.

In additional embodiments, a RNAi molecule can be active as a Dicer substrate, to be converted to a RNAi molecule that can be active in a RISC complex.

In some aspects, a RNAi molecule can have complementary guide and passenger sequence portions at opposing ends of a long molecule, so that the molecule can form a duplex region with the complementary sequence portions, and the strands are linked at one end of the duplex region by either nucleotide or non-nucleotide linkers. For example, a hairpin arrangement, or a stem and loop arrangement. The linker interactions with the strands can be covalent bonds or non-covalent interactions.

A RNAi molecule of this disclosure may include a nucleotide, non-nucleotide, or mixed nucleotide/non-nucleotide linker that joins the sense region of the nucleic acid to the antisense region of the nucleic acid. A nucleotide linker can be a linker of ≥2 nucleotides in length, for example about 3, 4, 5, 6, 7, 8, 9, or 10 nucleotides in length. The nucleotide linker can be a nucleic acid aptamer. By “aptamer” or “nucleic acid aptamer” as used herein refers to a nucleic acid molecule that binds specifically to a target molecule wherein the nucleic acid molecule has sequence that includes a sequence recognized by the target molecule in its natural setting. Alternately, an aptamer can be a nucleic acid molecule that binds to a target molecule, where the target molecule does not naturally bind to a nucleic acid. For example, the aptamer can be used to bind to a ligand-binding domain of a protein, thereby preventing interaction of the naturally occurring ligand with the protein. See, e.g., Gold et al., Annu Rev Biochem, 1995, Vol. 64, pp. 763-797; Brody et al., J. Biotechnol., 2000, Vol. 74, pp. 5-13; Hermann et al., Science, 2000, Vol. 287, pp. 820-825.

Examples of a non-nucleotide linker include an abasic nucleotide, polyether, polyamine, polyamide, peptide, carbohydrate, lipid, polyhydrocarbon, or other polymeric compounds, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc., 1991, Vol. 113, pp. 6324-6326; Jaeschke et al., Tetrahedron Lett., 1993, Vol. 34, pp. 301; Arnold et al., WO1989/002439; Usman et al., WO1995/006731; Dudycz et al., WO1995/011910, and Ferentz et al., J. Am. Chem. Soc., 1991, Vol. 113, pp. 4000-4002.

A RNAi molecule can have one or more overhangs from the duplex region. The overhangs, which are non-base-paired, single strand regions, can be from one to eight nucleotides in length, or longer. An overhang can be a 3′-end overhang, wherein the 3′-end of a strand has a single strand region of from one to eight nucleotides. An overhang can be a 5′-end overhang, wherein the 5′-end of a strand has a single strand region of from one to eight nucleotides.

The overhangs of a RNAi molecule can have the same length, or can be different lengths.

A RNAi molecule can have one or more blunt ends, in which the duplex region ends with no overhang, and the strands are base paired to the end of the duplex region.

A RNAi molecule of this disclosure can have one or more blunt ends, or can have one or more overhangs, or can have a combination of a blunt end and an overhang end.

A 5′-end of a strand of a RNAi molecule may be in a blunt end, or can be in an overhang. A 3′-end of a strand of a RNAi molecule may be in a blunt end, or can be in an overhang.

A 5′-end of a strand of a RNAi molecule may be in a blunt end, while the 3′-end is in an overhang. A 3′-end of a strand of a RNAi molecule may be in a blunt end, while the 5′-end is in an overhang.

In some embodiments, both ends of a RNAi molecule are blunt ends.

In additional embodiments, both ends of a RNAi molecule have an overhang.

The overhangs at the 5′- and 3′-ends may be of different lengths.

In certain embodiments, a RNAi molecule may have a blunt end where the 5′-end of the antisense strand and the 3′-end of the sense strand do not have any overhanging nucleotides.

In further embodiments, a RNAi molecule may have a blunt end where the 3′-end of the antisense strand and the 5′-end of the sense strand do not have any overhanging nucleotides.

A RNAi molecule may have mismatches in base pairing in the duplex region. The sense strand and the antisense strand of an siRNA can have mismatches in complementarity.

Any nucleotide in an overhang of a RNAi molecule can be a deoxyribonucleotide, or a ribonucleotide.

One or more deoxyribonucleotides may be at the 5′-end, where the 3′-end of the other strand of the RNAi molecule may not have an overhang, or may not have a deoxyribonucleotide overhang.

One or more deoxyribonucleotides may be at the 3′-end, where the 5′-end of the other strand of the RNAi molecule may not have an overhang, or may not have a deoxyribonucleotide overhang.

In some embodiments, one or more, or all of the overhang nucleotides of a RNAi molecule may be 2′-deoxyribonucleotides.

### Methods for Using RNAi Molecules

The nucleic acid molecules and RNAi molecules of this invention may be delivered to a cell or tissue by direct application of the molecules, or with the molecules combined with a carrier or a diluent.

The nucleic acid molecules and RNAi molecules of this invention can be delivered or administered to a cell, tissue, organ, or subject by direct application of the molecules with a carrier or diluent, or any other delivery vehicle that acts to assist, promote or facilitate entry into a cell, for example, viral sequences, viral material, or lipid or liposome formulations.

The nucleic acid molecules and RNAi molecules of this invention can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection.

Delivery systems may include, for example, aqueous and nonaqueous gels, creams, emulsions, microemulsions, liposomes, ointments, aqueous and nonaqueous solutions, lotions, aerosols, hydrocarbon bases and powders, and can contain excipients such as solubilizers and permeation enhancers.

Compositions and methods of this disclosure can include an expression vector that includes a nucleic acid sequence encoding at least one RNAi molecule of this invention in a manner that allows expression of the nucleic acid molecule.

The nucleic acid molecules and RNAi molecules of this invention can be expressed from transcription units inserted into DNA or RNA vectors. Recombinant vectors can be DNA plasmids or viral vectors. Viral vectors can be used that provide for transient expression of nucleic acid molecules.

For example, the vector may contain sequences encoding both strands of a RNAi molecule of a duplex, or a single nucleic acid molecule that is self-complementary and thus forms a RNAi molecule. An expression vector may include a nucleic acid sequence encoding two or more nucleic acid molecules.

A nucleic acid molecule may be expressed within cells from eukaryotic promoters. Those skilled in the art realize that any nucleic acid can be expressed in eukaryotic cells from the appropriate DNA/RNA vector.

In some aspects, a viral construct can be used to introduce an expression construct into a cell, for transcription of a dsRNA construct encoded by the expression construct.

Lipid formulations can be administered to animals by intravenous, intramuscular, or intraperitoneal injection, or orally or by inhalation or other methods as are known in the art.

Pharmaceutically acceptable formulations for administering oligonucleotides are known and can be used.

### Methods of Use of RNAi Molecules

The nucleic acid molecules and RNAi molecules of this invention may be delivered to a cell or tissue by direct application of the molecules, or with the molecules combined with a carrier or a diluent.

The nucleic acid molecules and RNAi molecules of this invention can be delivered or administered to a cell, tissue, organ, or subject by direct application of the molecules with a carrier or diluent, or any other delivery vehicle that acts to assist, promote or facilitate entry into a cell, for example, viral sequences, viral material, or lipid or liposome formulations.

The nucleic acid molecules and RNAi molecules of this invention can be complexed with one or more ionizable compounds, cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection.

Delivery systems may include, for example, aqueous and nonaqueous gels, creams, emulsions, microemulsions, liposomes, ointments, aqueous and nonaqueous solutions, lotions, aerosols, hydrocarbon bases and powders, and can contain excipients such as solubilizers and permeation enhancers.

A inhibitory nucleic acid molecule or composition of this invention may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracistemal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Compositions and methods of this disclosure can include an expression vector that includes a nucleic acid sequence encoding at least one RNAi molecule of this invention in a manner that allows expression of the nucleic acid molecule.

The nucleic acid molecules and RNAi molecules of this invention can be expressed from transcription units inserted into DNA or RNA vectors. Recombinant vectors can be DNA plasmids or viral vectors. Viral vectors can be used that provide for transient expression of nucleic acid molecules.

For example, the vector may contain sequences encoding both strands of a RNAi molecule of a duplex, or a single nucleic acid molecule that is self-complementary and thus forms a RNAi molecule. An expression vector may include a nucleic acid sequence encoding two or more nucleic acid molecules.

A nucleic acid molecule may be expressed within cells from eukaryotic promoters. Those skilled in the art realize that any nucleic acid can be expressed in eukaryotic cells from the appropriate DNA/RNA vector.

In some aspects, a viral construct can be used to introduce an expression construct into a cell, for transcription of a dsRNA construct encoded by the expression construct, where the dsRNA is active in RNA interference.

Lipid formulations can be administered to animals by intravenous, intramuscular, or intraperitoneal injection, or orally or by inhalation or other methods as are known in the art.

Pharmaceutically acceptable formulations for administering oligonucleotides are known and can be used.

In one embodiment of the above method, the inhibitory nucleic acid molecule is administered at a dosage of about 5 to 500 mg/m²/day, e.g., 5, 25, 50, 100, 125, 150, 175, 200, 225, 250, 275, or 300 mg/m²/day.

In some embodiments, the inhibitory nucleic acid molecules of this invention are administered systemically in dosages from about 1 to 100 mg/kg, e.g., 1, 5, 10, 20, 25, 50, 75, or 100 mg/kg.

In further embodiments, the dosage can range from about 25 to 500 mg/m²/day.

Methods known in the art for making formulations are found, for example, in “Remington: The Science and Practice of Pharmacy” Ed. A. R. Gennaro, Lippincourt Williams & Wilkins, Philadelphia, Pa., 2000.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleotide oligomer of the invention can depend on such variables as the type and extent of the disorder, the overall health status of the particular patient, the formulation of the compound excipients, and its route of administration.

All of the above methods for reducing viral titer may be either an in vitro method or an in vivo method. Dosage may be determined by an in vitro test using cultured cells, etc., as is known in the art. An effective amount may be an amount that reduces viral titer by at least 10%, at least 20%, or at least 30%, or at least 40%, or at least 50%, or at least 60%, or at least 70%, or at least 80%, or at least 90%, up to 100% of the tumor size.

### Formulations for Delivery of Nucleic Acid Agents

This invention can provide a composition for use in distributing an active agent in cells, tissues or organs, organisms, and subjects, where the composition includes one or more ionizable lipid molecules of this invention.

Compositions of this invention may include one or more of the ionizable lipid molecules, along with a structural lipid, one or more stabilizer lipids, and one or more lipids for reducing immunogenicity of the nanoparticles.

An ionizable lipid molecule of this invention can be any mol % of a composition of this invention.

The ionizable lipid molecules of a composition of this invention can be from 15 mol % to 40 mol % of the lipid components of the composition. In certain embodiments, the ionizable lipid molecules of a composition can be from 20 mol % to 35 mol % of the lipid components of the composition. In further embodiments, the ionizable lipid molecules of a composition can be from 25 mol % to 30 mol % of the lipid components of the composition.

The structural lipid of a composition of this invention can be from 25 mol % to 40 mol % of the lipid components of the composition. In certain embodiments, the structural lipid of a composition can be from 30 mol % to 35 mol % of the lipid components of the composition.

The sum of the stabilizer lipids of a composition of this invention can be from 25 mol % to 40% mol % of the lipid components of the composition. In certain embodiments, the sum of the stabilizer lipids of a composition can be from 30 mol % to 40 mol % of the lipid components of the composition.

In some embodiments, a composition of this invention can include two or more stabilizer lipids, where each of the stabilizer lipids individually can be from 5 mol % to 35 mol % of the lipid components of the composition. In certain embodiments, a composition of this invention can include two or more stabilizer lipids, where each of the stabilizer lipids individually can be from 10 mol % to 30 mol % of the lipid components of the composition.

In certain embodiments, the sum of the one or more stabilizer lipids can be from 25 mol % to 40 mol % of the lipids of the composition, wherein each of the stabilizer lipids individually can be from 5 mol % to 35% mol %.

In certain embodiments, the sum of the one or more stabilizer lipids can be from 30 mol % to 40 mol % of the lipids of the composition, wherein each of the stabilizer lipids individually can be from 10 mol % to 30% mol %.

The one or more lipids for reducing immunogenicity of the nanoparticles can be from a total of 1 mol % to 8 mol % of the lipid components of the composition. In certain embodiments, the one or more lipids for reducing immunogenicity of the nanoparticles can be from a total of 1 mol % to 5 mol % of the lipid components of the composition.

In additional aspects, a composition of this invention can further include a cationic lipid, which can be from 5 mol % to 25 mol % of the lipid components of the composition. In certain embodiments, a composition of this invention can further include a cationic lipid, which can be from 5 mol % to 15 mol % of the lipid components of the composition. In these aspects, the molar ratio of the concentrations of the cationic lipid to the ionizable lipid molecules of a composition of this invention can be from 5:35 to 25:15.

In compositions of this invention, the entirety of the lipid components may include one or more of the ionizable lipid molecular components, one or more structural lipids, one or more stabilizer lipids, and one or more lipids for reducing immunogenicity of the nanoparticles.

### Examples of Lipid Compositions

In some embodiments, three lipid-like components, i.e. one or more ionizable molecules, a structural lipid, and one or more lipids for reducing immunogenicity of the nanoparticles can be 100% of the lipid components of the composition. In certain embodiments, a cationic lipid can be included.

Examples of compositions of this invention are shown in Table 1.

TABLE 1
Compositions of lipid components (each in mol % of total)

| | | | Reduce |
|---|---|---|---|
| Ionizable | Cationic | Structural | immun. |
| 60 | 0 | 32 | 8 |
| 60 | 0 | 35 | 5 |
| 55 | 0 | 44 | 1 |
| 65 | 0 | 32 | 3 |
| 60 | 0 | 36 | 4 |
| 65 | 0 | 32 | 3 |
| 70 | 0 | 25 | 5 |
| 74 | 0 | 20 | 6 |
| 78 | 0 | 20 | 2 |
| 50 | 10 | 35 | 5 |
| 55 | 15 | 25 | 5 |
| 55 | 20 | 20 | 5 |

In certain embodiments, the four lipid-like components, i.e. one or more ionizable lipid molecules, a structural lipid, one or more stabilizer lipids, and one or more lipids for reducing immunogenicity of the nanoparticles, can be 100% of the lipid components of the composition.

Examples of compositions of this invention are shown in Table 2.

TABLE 2
Compositions of lipid components (each in mol % of total)

| | | | | Reduce |
|---|---|---|---|---|
| Ionizable | Cationic | Structural | Stabilizer | immun. |
| 17 | 0 | 35 | 40 | 8 |
| 20 | 0 | 35 | 40 | 5 |
| 25 | 0 | 35 | 39 | 1 |
| 25 | 0 | 35 | 35 | 5 |
| 25 | 0 | 30 | 40 | 5 |
| 25 | 0 | 40 | 30 | 5 |
| 30 | 0 | 25 | 40 | 5 |
| 35 | 0 | 25 | 35 | 5 |
| 40 | 0 | 30 | 25 | 5 |
| 25 | 5 | 30 | 35 | 5 |
| 25 | 10 | 30 | 30 | 5 |
| 25 | 15 | 25 | 30 | 5 |

### Ionizable Lipid-Like Molecules

Examples of on ionizable lipid include the following compound:

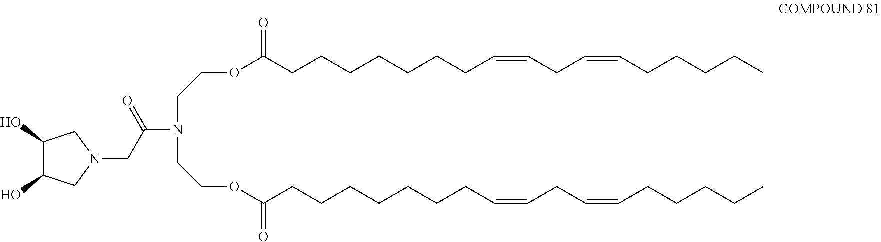 [SMILES: CCCCC/C=C\C/C=C\CCCCCCCC(=O)OCCN(CCOC(=O)CCCCCCC/C=C\C/C=C\CCCCC)C(=O)CN1C[C@@H](O)[C@@H](O)C1]

which is ((2-((3S,4R)-3,4-dihydroxypyrrolidin-1-yl)acetyl)azanediyl)bis(ethane-2,1-diyl) (9Z,9′Z,12Z,12′Z)-bis(octadeca-9,12-dienoate).

Examples of on ionizable lipid include the following compound:

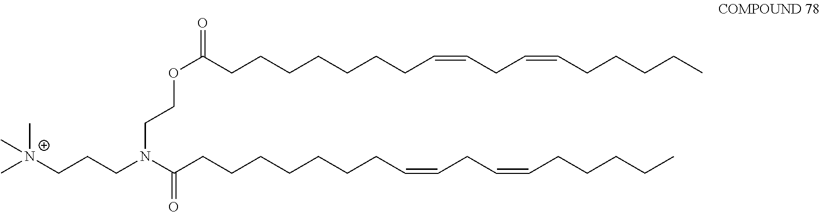 [SMILES: CCCCC/C=C\C/C=C\CCCCCCCC(=O)OCCN(CCC[N+](C)(C)C)C(=O)CCCCCCC/C=C\C/C=C\CCCCC]

which is N,N,N-trimethyl-3-((9Z,12Z)-N-(2-(((9Z,12Z)-octadeca-9,12-dienoyl)oxy)ethyl)octadeca-9,12-dienamido)propan-1-aminium.

Examples of an ionizable compound include the following compound:

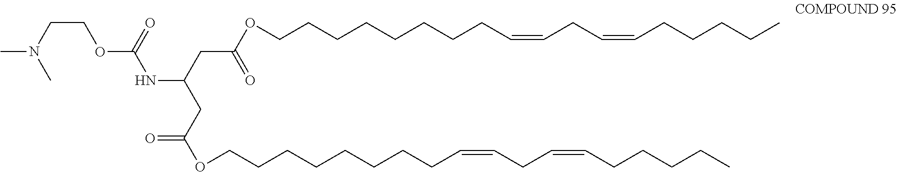 [SMILES: CCCCC/C=C\C/C=C\CCCCCCCCOC(=O)CC(CC(=O)OCCCCCCCC/C=C\C/C=C\CCCCC)NC(=O)OCCN(C)C]

which is di((9Z,12Z)-octadeca-9,12-dien-1-yl) 3-(((2-(dimethylamino)ethoxy)carbonyl)amino)pentanedioate.

Examples of an ionizable compound include the following compound:

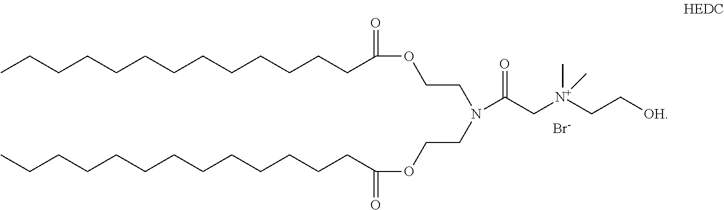 [SMILES: CCCCCCCCCCCCCC(=O)OCCN(CCOC(=O)CCCCCCCCCCCCC)C(=O)C[N+](C)(C)CCO ; [Br-]]

Examples of an ionizable compound include the following compound:

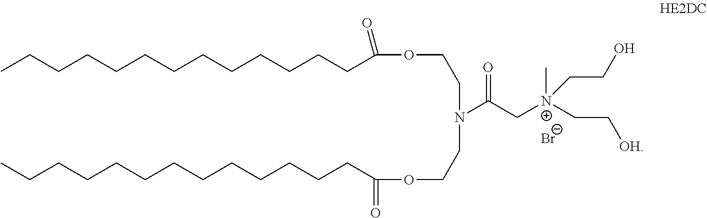 [SMILES: CCCCCCCCCCCCCC(=O)OCCN(CCOC(=O)CCCCCCCCCCCCC)C(=O)C[N+](C)(CCO)CCO ; [Br-]]

Examples of an ionizable compound include the following compound:

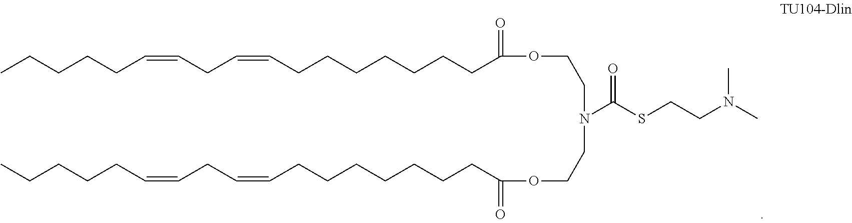 [SMILES: CCCCC/C=C\C/C=C\CCCCCCCC(=O)OCCN(CCOC(=O)CCCCCCC/C=C\C/C=C\CCCCC)C(=O)SCCN(C)C]

Examples of an ionizable compound include the following compound:

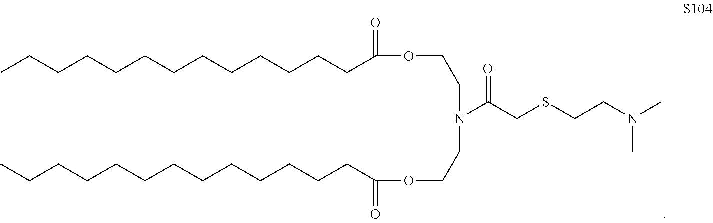 [SMILES: CCCCCCCCCCCCCC(=O)OCCN(CCOC(=O)CCCCCCCCCCCCC)C(=O)CSCCN(C)C]

### Structural Lipids

Examples of structural lipids include cholesterols, sterols, and steroids.

Examples of structural lipids include cholanes, cholestanes, ergostanes, campestanes, poriferastanes, stigmastanes, gorgostanes, lanostanes, gonanes, estranes, androstanes, pregnanes, and cycloartanes.

Examples of structural lipids include sterols and zoosterols such as cholesterol, lanosterol, zymosterol, zymostenol, desmosterol, stigmastanol, dihydrolanosterol, and 7-dehydrocholesterol.

Examples of structural lipids include pegylated cholesterols, and cholestane 3-oxo-(C1-22)acyl compounds, for example, cholesteryl acetate, cholesteryl arachidonate, cholesteryl butyrate, cholesteryl hexanoate, cholesteryl myristate, cholesteryl palmitate, cholesteryl behenate, cholesteryl stearate, cholesteryl caprylate, cholesteryl n-decanoate, cholesteryl dodecanoate, cholesteryl nervonate, cholesteryl pelargonate, cholesteryl n-valerate, cholesteryl oleate, cholesteryl elaidate, cholesteryl erucate, cholesteryl heptanoate, cholesteryl linolelaidate, and cholesteryl linoleate.

Examples of structural lipids include sterols such as phytosterols, beta-sitosterol, campesterol, ergosterol, brassicasterol, delta-7-stigmasterol, and delta-7-avenasterol.

### Stabilizer Lipids

Examples of stabilizer lipids include zwitterionic lipids.

Examples of stabilizer lipids include compounds such as phospholipids.

Examples of phospholipids include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine and ordilinoleoylphosphatidylcholine.

Examples of stabilizer lipids include phosphatidyl ethanolamine compounds and phosphatidyl choline compounds.

Examples of stabilizer lipids include 1,2-Dioleoyl-sn-Glycero-3-Phosphocholine (DOPC).

Examples of stabilizer lipids include diphytanoyl phosphatidyl ethanolamine (DPhPE) and 1,2-Diphytanoyl-sn-Glycero-3-Phosphocholine (DPhPC).

Examples of stabilizer lipids include 1,2-distearoyl-sn-glycero-3-phosphocholine (DSPC), 1,2-dipalmitoyl-sn-glycero-3-phosphocholine (DPPC), 1,2-dipalmitoyl-sn-glycero-3-phosphoethanolamine (DPPE), and 1,2-dioleoyl-sn-glycero-3-phosphoethanolamine (DOPE).

Examples of stabilizer lipids include 1,2-dilauroyl-sn-glycerol (DLG); 1,2-dimyristoyl-sn-glycerol (DMG); 1,2-dipalmitoyl-sn-glycerol (DPG); 1,2-distearoyl-sn-glycerol (DSG); 1,2-diarachidoyl-sn-glycero-3-phosphocholine (DAPC); 1,2-dilauroyl-sn-glycero-3-phosphocholine (DLPC); 1,2-dimyristoyl-sn-glycero-3-phosphocholine (DMPC); 1,2-dipalmitoyl-sn-glycero-O-ethyl-3-phosphocholine (DPePC); 1,2-dilauroyl-sn-glycero-3-phosphoethanolamine (DLPE); 1,2-dimyristoyl-sn-glycero-3-phosphoethanolamine (DMPE); 1,2-distearoyl-sn-glycero-3-phosphoethanolamine (DSPE); 1-palmitoyl-2-linoleoyl-sn-glycero-3-phosphocholine; 1-palmitoyl-2-oleoyl-sn-glycero-3-phosphocholine (POPC); 1-palmitoyl-2-lyso-sn-glycero-3-phosphocholine (P-Lyso-PC); and 1-Stearoyl-2-lyso-sn-glycero-3-phosphocholine (S-Lyso-PC).

### Lipids for Reducing Immunogenicity

Examples of lipids for reducing immunogenicity include polymeric compounds and polymer-lipid conjugates.

Examples of lipids for reducing immunogenicity include pegylated lipids having polyethyleneglycol (PEG) regions. The PEG regions can be of any molecular mass. In some embodiments, a PEG region can have a molecular mass of 200, 300, 350, 400, 500, 550, 750, 1000, 1500, 2000, 3000, 3500, 4000 or 5000 Da.

Examples of lipids for reducing immunogenicity include compounds having a methoxypolyethyleneglycol region.

Examples of lipids for reducing immunogenicity include compounds having a carbonyl-methoxypolyethyleneglycol region.

Examples of lipids for reducing immunogenicity include compounds having a multi-branched PEG region.

Examples of lipids for reducing immunogenicity include compounds having a polyglycerine region.

Examples of lipids for reducing immunogenicity include polymeric lipids such as DSPE-mPEG, DMPE-mPEG, DPPE-mPEG, and DOPE-mPEG.

Examples of lipids for reducing immunogenicity include PEG-phospholipids and PEG-ceramides.

### Cationic Lipids

Examples of cationic lipids include cationic HEDC compounds as described in US 2013/0330401 A1. Some examples of cationic lipids are given in US 2013/0115274 A1. Additional examples of cationic lipids are known in the art.

### Lipid Compositions

In some embodiments, a composition can contain the ionizable lipid compound 81, the structural lipid cholesterol, the stabilizer lipids DOPC and DOPE, and the lipid for reducing immunogenicity DPPE-mPEG. In certain embodiments, compound 81 can be 15 to 25 mol % of the composition; the cholesterol, DOPC, and DOPE combined can be 75 to 85 mol % of the composition; and DPPE-mPEG can be 5 mol % of the composition.

In one embodiment, compound 81 can be 25 mol % of the composition; cholesterol can be 30 mol % of the composition, DOPC can be 20 mol % of the composition, DOPE can be 20 mol % of the composition; and DPPE-mPEG(2000) can be 5 mol % of the composition.

### Nanoparticles

Embodiments of this invention can provide liposome nanoparticle compositions. The ionizable molecules of this invention can be used to form liposome compositions, which can have a bilayer of lipid-like molecules.

A nanoparticle composition can have one or more of the ionizable molecules of this invention in a liposomal structure, a bilayer structure, a micelle, a lamellar structure, or a mixture thereof.

In some embodiments, a composition can include one or more liquid vehicle components. A liquid vehicle suitable for delivery of active agents of this invention can be a pharmaceutically acceptable liquid vehicle. A liquid vehicle can include an organic solvent, or a combination of water and an organic solvent.

Embodiments of this invention can provide lipid nanoparticles having a size of from 10 to 1000 nm. In some embodiments, the liposome nanoparticles can have a size of from 10 to 150 nm.

In certain embodiments, the liposome nanoparticles of this invention can encapsulate the RNAi molecule and retain at least 80% of the encapsulated RNAi molecules after 1 hour exposure to human serum.

### Pharmaceutical Compositions

This invention further contemplates methods for distributing an active agent to an organ of a subject for treating malignant tumor by administering to the subject a composition of this invention. Organs that can be treated include lung, liver, pancreas, kidney, colon, bone, skin, and intestine.

In some embodiments, this invention provides methods for treating a lung malignant tumor disease by administering to the subject a composition of this invention.

In further aspects, this invention provides a range of pharmaceutical formulations.

A pharmaceutical formulation herein can include an active agent, as well as a drug carrier, or a lipid of this invention, along with a pharmaceutically acceptable carrier or diluent. In general, active agents of this description include any active agents for malignant tumor, including any inhibitory nucleic acid molecules and any small molecular drugs. Examples of inhibitory nucleic acid molecules include ribozymes, anti-sense nucleic acids, and RNA interference molecules (RNAi molecules).

A drug carrier may target a composition to reach stellate cells. A drug carrier may include a drug in its interior, or be attached to the exterior of a drug-containing substance, or be mixed with a drug so long as a retinoid derivative and/or vitamin A analogue is included in the drug carrier, and is at least partially exposed on the exterior of the preparation. The composition or preparation may be covered with an appropriate material, such as, for example, an enteric coating or a material that disintegrates over time, or may be incorporated into an appropriate drug release system.

A pharmaceutical formulation of this invention may contain one or more of each of the following: a surface active agent, a diluent, an excipient, a preservative, a stabilizer, a dye, and a suspension agent.

Some pharmaceutical carriers, diluents and components for a pharmaceutical formulation, as well as methods for formulating and administering the compounds and compositions of this invention are described in Remington's Pharmaceutical Sciences, 18th Ed., Mack Publishing Co., Easton, Pa. (1990).

Examples of preservatives include sodium benzoate, ascorbic acid, and esters of p-hydroxybenzoic acid.

Examples of surface active agents include alcohols, esters, sulfated aliphatic alcohols.

Examples of excipients include sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, and calcium carboxymethyl cellulose.

Examples of suspension agents include coconut oil, olive oil, sesame oil, peanut oil, soya, cellulose acetate phthalate, methylacetate-methacrylate copolymer, and ester phthalates.

A therapeutic formulation of this invention for the delivery of one or more molecules active for gene silencing can be administered to a mammal in need thereof. A therapeutically effective amount of the formulation and active agent, which may be encapsulated in a liposome, can be administered to a mammal for preventing or treating malignant tumor.

The route of administration may be local or systemic.

A therapeutically-effective formulation of this invention can be administered by various routes, including intravenous, intraperitoneal, intramuscular, subcutaneous, and oral.

Routes of administration may include, for example, parenteral delivery, including intramuscular, subcutaneous, intravenous, intramedullary injections, as well as intrathecal, direct intraventricular, intraperitoneal, intranasal, or intraocular injections.

The formulation can also be administered in sustained or controlled release dosage forms, including depot injections, osmotic pumps, and the like, for prolonged and/or timed, pulsed administration at a predetermined rate.

The composition of the present invention may be administered via various routes including both oral and parenteral routes, and examples thereof include, but are not limited to, oral, intravenous, intramuscular, subcutaneous, local, intrapulmonary, intra-airway, intratracheal, intrabronchial, nasal, rectal, intraarterial, intraportal, intraventricular, intramedullar, intra-lymph-node, intralymphatic, intrabrain, intrathecal, intracerebroventricular, transmucosal, percutaneous, intranasal, intraperitoneal, and intrauterine routes, and it may be formulated into a dosage form suitable for each administration route. Such a dosage form and formulation method may be selected as appropriate from any known dosage forms and methods. See e.g. Hyojun Yakuzaigaku, Standard Pharmaceutics, Ed. by Yoshiteru Watanabe et al., Nankodo, 2003.

Examples of dosage forms suitable for oral administration include, but are not limited to, powder, granule, tablet, capsule, liquid, suspension, emulsion, gel, and syrup, and examples of the dosage form suitable for parenteral administration include injections such as an injectable solution, an injectable suspension, an injectable emulsion, and a ready-to-use injection. Formulations for parenteral administration may be a form such as an aqueous or nonaqueous isotonic sterile solution or suspension.

Pharmaceutical formulations for parenteral administration, e.g., by bolus injection or continuous infusion, include aqueous solutions of the active formulation in water-soluble form. Suspensions of the active compounds may be prepared as appropriate oily injection suspensions. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Formulations for injection may be presented in unit dosage form, e.g., in ampoules or in multi-dose containers, with an added preservative. The formulations may take such forms as suspensions, solutions or emulsions in oily or aqueous vehicles, and may contain formulary agents such as suspending, stabilizing and/or dispersing agents. Alternatively, the active ingredient may be in powder form for constitution with a suitable vehicle, e.g., sterile pyrogen-free water, before use.

In addition to the preparations described previously, the formulations may also be formulated as a depot preparation. Such long acting formulations may be administered by intramuscular injection. Thus, for example, the formulation may be formulated with suitable polymeric or hydrophobic materials, for example as an emulsion in an acceptable oil, or ion exchange resins, or as sparingly soluble derivatives, for example, as a sparingly soluble salt.

Compositions and formulations of this invention may also be formulated for topical delivery and may be applied to the subject's skin using any suitable process for application of topical delivery vehicle. For example, the formulation may be applied manually, using an applicator, or by a process that involves both. Following application, the formulation may be worked into the subject's skin, e.g., by rubbing. Application may be performed multiple times daily or on a once-daily basis. For example, the formulation may be applied to a subject's skin once a day, twice a day, or multiple times a day, or may be applied once every two days, once every three days, or about once every week, once every two weeks, or once every several weeks.

The formulations or pharmaceutical compositions described herein may be administered to the subject by any suitable means. Examples of methods of administration include, among others, (a) administration via injection, subcutaneously, intraperitoneally, intravenously, intramuscularly, intradermally, intraorbitally, intracapsularly, intraspinally, intrasternally, or the like, including infusion pump delivery; (b) administration locally such as by injection directly in the renal or cardiac area, e.g., by depot implantation; as well as deemed appropriate by those of skill in the art for bringing the active compound into contact with living tissue.

The exact formulation, route of administration and dosage for the pharmaceutical compositions can be chosen by the individual physician in view of the patient's condition. See, e.g., Goodman & Gilman's The Pharmacological Basis of Therapeutics, 12^{th} Ed., Sec. 1, 2011. Typically, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some condition, the dosages will be about the same, or dosages that are about 0.1% to about 500%, more preferably about 25% to about 250% of the established human dosage. Where no human dosage is established, as will be the case for newly-discovered pharmaceutical compositions, a suitable human dosage can be inferred from ED50 or ID50 values, or other appropriate values derived from in vitro or in vivo studies, as qualified by toxicity studies and efficacy studies in animals.

### Example Protocol

Example protocol for activity screening using psiCHECK reporter assay. The psiCHECK2-EBO reporter construct contains all siRNA targeting regions in sequential order with about 10 bp upstream and downstream of siRNA target sequence. Experimental daily schedule: Day 1, Cell seeding at 5˜7.5×10³/100 ul/well; Day 2, Co-transfection (cell confluence around 80%); Day 3, Cell harvest for luciferase activity measurement.
Platform: 96 wells plate
# of wells/sample: Triplicate
[siRNA]: 5 pM, 50 pM & 500 pM
Cell line: HeLa
Incubation medium DMEM (containing 10% FBS)
Transfection agent: Lipofectamine 2000 (L2K)
Incubation condition: At 37° C. incubator under 5% CO₂
Harvest time: 24 hours after transfection
Reporter Assay Kit: Dual-Luciferase Reporter Assay (Promega, Cat #: E1960)

### Example Co-Transfection Procedure for 50 pM siRNA Concentration

1. Dilute 0.1 μL of 50 nM siRNA (final conc. 50 pM) in 2.9 μL Opti-MEM.

2. Dilute 0.1 uL of 100 ng/mL psiCHECK plasmid in 2.9 uL Opti-MEM.

3. Dilute 0.1 uL L2K in 3.9 μL Opti-MEM.

4. Combine the diluted siRNA and psiCHECK plasmid with the diluted L2K. Mix gently by tapping without pipetting and incubate for 15 min at RT.

5. During incubation aspirate culture media on dish and then replace with 90 μL DMEM.

6. Add the siRNA-plasmid-L2K complex drop by drop to a plate. This gives a final volume of 100 μL and a final siRNA concentration of 50 pM.

7. Mix gently by rocking the dish back and forth.

8. Incubate the cells overnight at 37° C. in a CO₂ incubator.

9. Measure luciferase activity using Promega's Luciferase Assay System (Promega, E4550) according to manufacturer protocol.

### Example Sequential Transfection Protocol to Determine siRNA Liposomal Formulation Knockdown Activity with psiCHECK Reporter System

Platform: 96 wells plate
# of wells/sample: Duplicate
[siRNA] 5000 to 0.05243 nM (diluted in D-PBS, 1×) 2.5 fold serial dilutions
Incubation medium DMEM (containing 10% FBS)
Transfection agent: Lipofectamine 2000 (L2K)
Incubation condition: At 37° C. incubator under 5% CO2
Harvest time: 24 hours after transfection

### Transfection Procedure

1. Dilute EBO psiCHECK plasmid in Opti-MEM.

2. Dilute L2K in Opti-MEM.

3. Combine the diluted psiCHECK plasmid with the diluted L2K. Mix gently by tapping without pipetting and incubate for 5 min at RT.

4. During incubation aspirate culture media on dish, rinse twice with PBS and then replace with 90 μL full DMEM culture medium.

5. Add 10 ul of plasmid/L2K complex drop by drop to a plate, giving the final volume of 100 μL.

6. Mix gently by rocking the dish.

7. Incubate the cells overnight at 37° C. in a CO₂ incubator.

8. Measure luciferase activity using Promega's Luciferase Assay System (Promega, E4550) according to manufacturer protocol.

Procedure: plate HeLa cells and incubate; transfect cells with reporter construct using Lipofectamine; remove medium with formulation (optional wash step); transfect cells with siRNA in formulation and incubate for 24 h; measure Luciferase activity.

### EXAMPLES

### Example 1

A formulation including siRNAs targeted to Ebola virus mRNAs encoding ZEBOV proteins is administered in rhesus macaques (n=6). In this example, a combination of inventive siRNA molecules is delivered to the rhesus macaques, which target Ebola L polymerase, VP24, and VP35 genes. After exposure to virus, an effective amount of the formulation is delivered once per week for four weeks. After this administration, two-thirds of the rhesus monkeys survive a lethal ZEBOV infection challenge. In a second trial, after exposure to virus, an effective amount of the formulation is delivered once per week for eight weeks. After this administration, all of the rhesus monkeys survive a lethal ZEBOV infection challenge.

### Example 2

A siRNA formulation of this invention is injected in the tail vein of six to eight-week-old female CD1 ICR mice. Cytokine induction is measured in blood and liver tissue collected 4 h after siRNA injection.

### Example 3

158 Ebola strains were utilized to determine 39 Ebola siRNA sequences, all of which show 100% homology to the 2014 Ebola outbreak data (102 strains). 21 of these sequences have high homology, from 88% to 100%, across all strains. Another selected group of sequences target two different regions in the genome. The sequences were selected for having reduced off target hits in the human RefSeq RNA database. Published sequences are used as positive controls.

### Example 4

Examples of formulations of this invention include those in Table 3.

TABLE 3
Lipid nanoparticle formulations

| No. | Lipid Description | Lipid Ratio |
|---|---|---|
| 1 | HEDC:S104:CH:DOPE:DMPE-PEG-2K with | 20:20:25:30:2 |
| | (siRNA) | |
| 2 | HEDC:S104:CH:DOPE:DMPE-PEG-2K:diVA | 20:20:26:30:2:2 |
| | with (siRNA) | |

wherein HEDC and S104 are as disclosed in US 2013/0022665 A1, “CH” refers to cholesterol, DOPE is 1,2-Dioleoyl-sn-Glycero-3-Phosphoethanolamine, DMPE-PEG2K is N-(Carbonyl-methoxypolyethyleneglycol 2000)-1,2-dimyristoyl-sn-glycero-3-phosphoethanolamine, and diVA is a Vitamin A excipient.

The formulations of Table 3 are characterized in Table 4.

TABLE 4
Characterization of lipid nanoparticle formulations

| Particle Size | | | | | | | |
|---|---|---|---|---|---|---|---|
| | Vol. | | | | | Zeta | siRNA |
| Z-ave | mean | | D(v) | D(v) | D(v) | Potential | Content % |
| (nm) | (nm) | PDI | 10 | 50 | 90 | (mV) | encapsulated |
| 71 | 61 | 0.117 | 39 | 56 | 89 | 1.8 | 99 |
| 72 | 62 | 0.093 | 40 | 58 | 92 | 4.0 | 98 |

### Example 5

Examples of siRNAs of this invention include those in Table 5. All of the example non-control siRNAs in Table 5 had 100% homology to the 102 strains of the 2014 Ebola outbreak in western Africa.

TABLE 5
Ebola siRNA sequences, targets, and controls

| | Gene | Sense | Antisense |
|---|---|---|---|
| Pos. | Region | SEQ ID NOs: 1-25 | SEQ ID NOs: 26-50 |
| 3031 | VP35 | UGAUGAAGAUUAAAACCUUmUmU | AAGGUUUUAAUCUUCAUCAmUmU |
| 4389/ | VP35/VP40 | UGAUGAAGAUUAAGAAAAAmUmU | UUUUUCUUAAUCUUCAUCAmUmU |
| 8287 | and | | |
| | GP/VP30 | | |
| 3033 | VP35 | AUGAAGAUUAAAACCUUCAmUmU | UGAAGGUUUUAAUCUUCAUmUmU |
| 3032 | VP35 | GAUGAAGAUUAAAACCUUCmUmU | GAAGGUUUUAAUCUUCAUCmUmU |
| 3035 | VP35 | GAAGAUUAAAACCUUCAUCmUmU | GAUGAAGGUUUUAAUCUUCmUmU |
| 3034 | VP 35 | UGAAGAUUAAAACCUUCAUmUmU | AUGAAGGUUUUAAUCUUCAmUmU |
| 11500 | VP24/L | UGAGGAAGAUUAAGAAAAAmUmU | UUUUUCUUAAUCUUCCUCAmUmU |
| 8286 | GP/VP30 | UUGAUGAAGAUUAAGAAAAmUmU | UUUUCUUAAUCUUCAUCAAmUmU |
| 8288 | GP/VP30 | GAUGAAGAUUAAGAAAAAGmUmU | CUUUUUCUUAAUCUUCAUCmUmU |
| 3030 | VP35 | AUGAUGAAGAUUAAAACCUmUmU | AGGUUUUAAUCUUCAUCAUmUmU |
| 3036 | VP35 | AAGAUUAAAACCUUCAUCAmUmU | UGAUGAAGGUUUUAAUCUUmUmU |
| 15524 | L | UUUUCCAGAAUGUUAUAAAmUmU | UUUAUAACAUUCUGGAAAAmUmU |
| 15523 | L | AUUUUCCAGAAUGUUAUAAmUmU | UUAUAACAUUCUGGAAAAUmUmU |
| 15526 | L | UUCCAGAAUGUUAUAAAUUmUmU | AAUUUAUAACAUUCUGGAAmUmU |
| 15525 | L | UUUCCAGAAUGUUAUAAAUmUmU | AUUUAUAACAUUCUGGAAAmUmU |
| 3037 | VP35 | AGAUUAAAACCUUCAUCAUmUmU | AUGAUGAAGGUUUUAAUCUmUmU |
| 16300 | L | CCUUUAUGGAUAGUAUAUCmUmU | GAUAUACUAUCCAUAAAGGmUmU |
| 13465 | L | UUUGUAACUGAUUUAGAGAmUmU | UCUCUAAAUCAGUUACAAAmUmU |
| 13466 | L | UUGUAACUGAUUUAGAGAAmUmU | UUCUCUAAAUCAGUUACAAmUmU |
| 3025/ | NP/VP35 | AACUAAUGAUGAAGAUUAAmUmU | UUAAUCUUCAUCAUUAGUUmUmU |
| 9878 | and VP24 | | |
| 3024/ | NP/VP35 | AAACUAAUGAUGAAGAUUAmUmU | UAAUCUUCAUCAUUAGUUUmUmU |
| 9877 | and VP24 | | |
| 11044 | Control | UCCUCGACACGAAUGCAAAmUmU | UUUGCAUUCGUGUCGAGGAmUmU |
| | VP24 | | |
| 3886 | Control | GCAAUUCAUUGGACAUUAUmUmU | AUAAUGUCCAAUGAAUUGCmUmU |
| | VP35 | | |
| 17397 | Control | GUACGAAGCUGUACAUAAAmUmU | UUUAUGUACAGCUUCGUACmUmU |
| | L | | |
| | Control | GAUUAUGUCCGGUUAUGUAmUmU | UACAUAACCGGACAUAAUCmUmU |
| | Luc | | |

In Table 5, Pos. refers to the gene position in Zaire ebolavirus isolate Ebola virus/H. sapiens-wt/SLE/2014/Makona-G3686.1, complete genome, Acc #KM034562.1. The Score is the sequence homology as compared to all 158 known and sequenced strains of Ebola virus.

In Table 5, positive controls are: position 3886, VP35, SEQ ID NOs:23 and 48; position 11044, VP24, SEQ ID NOs:22 and 47; and position 17397, L, SEQ ID NOs:24 and 49. See Geisbert T W, et al., Postexposure protection of non-human primates against a lethal Ebola virus challenge with RNA interference: a proof-of-concept study, Lancet, 2010 May 29; 375(9729):1896-905.

In Table 5, an inventive siRNA can be formed from any one of sense strands SEQ ID NOs:1-21 and the corresponding complementary antisense strand to the right in Table 5, being one of SEQ ID NOs:26-46.

### Example 6

Ebola siRNAs of this invention were found to be active for ebola gene silencing in a liposomal formulation in vitro. The dose-dependent activities of ebola siRNAs for gene knockdown were found to exhibit an IC50 below about 4 nM.

The activity of a siRNA of this invention (SEQ ID NOs:2 and 27, Position 389) in a liposomal formulation was measured with the psiCHECK reporter assay in a sequential transfection protocol. The IC50s of the siRNA formulations are shown in Table 6.

TABLE 6
Activity of Ebola siRNA formulations (IC50)

| | | | | DMPE- | | | |
|---|---|---|---|---|---|---|---|
| Ionizable compound | Chol | DOPE | DSPC | PEG-2K | diVA | | IC50 |
| Mol % | mol % | mol % | mol % | mol % | mol % | N/P | (nM) |
| HEDC:S104 | 25 | 30 | | 5 | 2 | 3 | 1.8 |
| 20:20 mol % | | | | | | | |
| HEDC:TU104Dlin | 25 | 30 | | 5 | 2 | 3 | 2.0 |
| 20:20 mol % | | | | | | | |
| HE2DC:TU104Dlin | 25 | 30 | | 5 | 2 | 3 | 1.6 |
| 20:20 mol % | | | | | | | |
| Compound 95 | 28 | 20 | | 2 | | 3 | 2.4 |
| 50 mol % | | | | | | | |
| Compound 81 | 36 | | 10 | 2 | 2 | 3 | 2.8 |
| 50 mol % | | | | | | | |
| Compound 81 | 38 | | 10 | 2 | | 3 | 3.9 |
| 50 mol % | | | | | | | |
| Compound 78 | 28 | 20 | | 2 | | 3 | 2.2 |
| 50 mol % | | | | | | | |

wherein HEDC and S104 are as disclosed in US 2013/0022665 A1, “Chol” refers to cholesterol, DOPE is 1,2-Dioleoyl-sn-Glycero-3-Phosphoethanolamine, DSPC is 1,2-distearoyl-sn-glycero-3-phosphocholine, DMPE-PEG2K is N-(Carbonyl-methoxypolyethyleneglycol 2000)-1,2-dimyristoyl-sn-glycero-3-phosphoethanolamine, and diVA is a Vitamin A excipient.

As shown in Table 6, the activities of the ebola siRNAs of this disclosure were very high, in the range of 1-4 nM, which is suitable for many uses, including as a drug agent to be used in vivo.

The embodiments described herein are not limiting and one skilled in the art can readily appreciate that specific combinations of the modifications described herein can be tested without undue experimentation toward identifying nucleic acid molecules with improved RNAi activity.

All publications, patents and literature specifically mentioned herein are incorporated by reference in their entirety for all purposes.

It is understood that this invention is not limited to the particular methodology, protocols, materials, and reagents described, as these may vary. It is also to be understood that the terminology used herein is for the purpose of describing particular embodiments only, and is not intended to limit the scope of the present invention. It will be readily apparent to one skilled in the art that varying substitutions and modifications can be made to the description disclosed herein without departing from the scope and spirit of the description, and that those embodiments are within the scope of this description and the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprises,” “comprising”, “containing,” “including”, and “having” can be used interchangeably, and shall be read expansively and without limitation.

Recitation of ranges of values herein are merely intended to serve as a shorthand method of referring individually to each separate value falling within the range, unless otherwise indicated herein, and each separate value is incorporated into the specification as if it were individually recited herein. For Markush groups, those skilled in the art will recognize that this description includes the individual members, as well as subgroups of the members of the Markush group.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

All of the features disclosed in this specification may be combined in any combination. Each feature disclosed in this specification may be replaced by an alternative feature serving the same, equivalent, or similar purpose.

1. A composition for inhibiting Ebola virus comprising one or more siRNAs targeted to at least one Ebola virus gene selected from NP, VP30, VP35, L polymerase, glycoprotein (GP), VP24, VP40, and intergenic overlapping regions selected from NP/VP35, VP35/VP40, GP/VP30, and VP24/L.

2. The composition of claim 1, wherein the one or more siRNAs are targeted to at least two Ebola genes selected from NP, VP30, VP35, L polymerase, glycoprotein (GP), VP24, VP40, and intergenic overlapping regions selected from NP/VP35, VP35/VP40, GP/VP30, and VP24/L.

3. The composition of claim 1, wherein at least one of the siRNAs comprises a sense strand having the sequence UGAUGAAGAUUAAGAAAAAmUmU SEQ ID NO:2 and an antisense strand having the sequence UUUUUCUUAAUCUUCAUCAmUmU SEQ ID NO:27.

4. The composition of claim 1, wherein the siRNAs each comprise a sense strand and an antisense strand, the sense strand having a sequence being any one of SEQ ID NOs:1-21 and the antisense strand having a sequence being complementary to the sense strand and being one of SEQ ID NOs:26-46.

5. The composition of claim 1, wherein at least one of the siRNAs inhibits an Ebola virus gene with an IC50 of less than 10 nM.

6. The composition of claim 1, wherein a single administration of at least one of the siRNAs inhibits Ebola virus titer levels by at least 25% in vivo.

7. The composition of claim 1, wherein the siRNAs each comprise 19-21 nucleotides in length.

8. The composition of claim 1, wherein the siRNAs comprise one or more modified, non-natural, or chemically-modified nucleotides.

9. The composition of claim 1, wherein the siRNAs comprise three or more 2′-OMe modified nucleotides.

10. The composition of claim 1, wherein the siRNAs comprise three to eight 2′-OMe modified nucleotides.

11. The composition of claim 1, wherein the siRNAs comprise a 3′ overhang or a blunt end.

12. A pharmaceutical composition comprising a composition of claim 1 and a delivery vehicle.

13. The composition of claim 12, wherein the delivery vehicle comprises liposomes and the siRNAs are encapsulated in the liposomes.

14. The composition of claim 12, wherein the delivery vehicle comprises an ionizable lipid, a structural lipid, one or more stabilizer lipids, and a lipid for reducing immunogenicity.

15. The composition of claim 12, wherein the ionizable compound is selected from compound 81, compound 78, compound 95, HEDC, 5104, HE2DC, TU104Dlin, and mixtures thereof.

16. A method for inhibiting Ebola virus gene expression in a cell, the method comprising contacting the cell with a composition of claim 12.

17. A method for inhibiting Ebola virus gene expression in a mammal in need thereof, the method comprising administering to the mammal a composition of claim 12.

18. A method for preventing, treating or ameliorating one or more symptoms of Ebola virus infection in a mammal in need thereof, the method comprising administering to the mammal a therapeutically effective amount of a composition of claim 12.

19. A method for preventing, treating or ameliorating one or more symptoms of Ebola virus infection in a mammal in need thereof, the method comprising administering to the mammal a therapeutically effective amount of a composition of claim 12, wherein after administration the Ebola viral titer of the mammal is reduced by at least 25%.

20. A method for inhibiting the replication of Ebola virus in a mammal in need thereof, the method comprising administering to the mammal a therapeutically effective amount of a composition of claim 12.
